(19) United States
(12) Patent Application Publication
(10) Pub. No.: US 2006/0286054 A1
(43) Pub. Date: Dec. 21, 2006

(54) PHARMACEUTICAL COMPOSITIONS FOR THE TREATMENT OF PSORIASIS

(75) Inventor: Hector J. Gomez, Lutz, FL (US)

Correspondence Address: PATREA L. PABST;PABST PATENT GROUP LLP, 400 COLONY SQUARE, SUITE 1200, ATLANTA, GA, 30361, US

(73) Assignee: Apollo Pharmaceutical, Inc.

(21) Appl. No.: 11/420,642

(22) Filed: May 26, 2006

Related U.S. Application Data
(60) Provisional application No. 60/690,990, filed on Jun. 15, 2005.

(51) Int. Cl. A61K 31/7034; A61K 31/59; A61K 31/4178; A61K 31/366

(52) U.S. Cl. 424/70.1; 514/167; 514/35; 514/397; 514/454

(57) ABSTRACT

Pharmaceutical compositions for the treatment of skin disorders such as psoriasis, acne and eczema, methods of making the compositions and methods of use thereof are described herein. The composition comprises psorberine, an alcohol-water extract isolated from the Mahonia aquifolium plant, and one or more additional active agents. In a preferred embodiment, the one or more active agents is a vitamin D3 analog, such as calcipotriol. The compositions may also contain excipients such as emollients, surfactants, emulsifiers and buffers. The compositions are formulated into a cream, lotion or ointment for topical administration.

### CROSS-REFERENCE TO RELATED APPLICATIONS

Priority is claimed to U.S. provisional application Serial No. 60/690,990 filed 15 Jun. 2005.

This invention is generally in the field of pharmaceutical compositions for the treatment of skin disorders such as psoriasis, acne, rosacea, eczema (atopic dermatitis) and other types of dermatitis (eg, contact dermatitis, dyshidrotic eczema, nummular dermatitis, seborrheic dermatitis), verruca vulgaris, tuberous sclerosis, pyogenic granulomas, recessive dystrophic epidermolysis bullosa, venous ulcers, molluscum contagious, seborrheic keratosis, and actinic keratosis.

### BACKGROUND OF THE INVENTION

Psoriasis is a chronic skin disease that is characterized by scaling and inflammation of the skin. When psoriasis develops, patches of skin thicken, redden, and become covered with silvery scales. These patches are generally referred to as plaques. The plaques are usually itchy and can burn. Psoriasis most often occurs on the elbows, knees, scalp, lower back, face, palms and soles of the feet. The scaling occurs when the cells in the outer layer of the skin reproduce faster that normal and accumulate on the skin's surface. Psoriasis affects about 1% to 3% of the North American population. It occurs in all age groups and affects men and women equally. People affected by psoriasis suffer from discomfort, restricted joint motion and emotional distress. About 10% of people sufffering from psoriasis have joint inflammation that produces symptoms similar to arthritis.

A variety of treatments and methods have been used over the years including the topical application of corticosteroids; vitamin D3 analogs such as calcipotriene; coal tar, etc. Bath solutions and general moisturizers have been utilized by some patients. Sunlight and ultraviolet light treatments have also been used. Systemic treatment with retinoids, methotrexate, cyclosporine, hydroxyurea and antibiotics is sometimes required. More recently, new biologic agents and biologic-immune-response modifiers such as alefacept, efalizumab, and etanercept have been developed.

Each of these treatments has its benefits and drawbacks. In many instances, patients develop a tolerance to the treatment resulting in decreased effectiveness. In addition, these treatments are often messy, have an unpleasant odor, and are repetitive and tedious for patients.

Topical treatments have included administration of corticosteroids, lotions, and a variety of other agents including an extract from the Mahonia aquifolium plant. U.S. Published patent application Ser. No. 20050069576 by Mills et al. describes a skin treatment composition comprising a Mahonia aquifoliumextract in a liposome delivery system. The Mahonia aquifolium extract is present in the skin treatment composition in a range of from 5% to 20% by weight of the total composition.

U.S. Published patent application Ser. No. 20010000731 to Qi et al. describes methods for the prevention and treatment of chronic venous insufficiency by application of an effective amount of an isoquinoline alkaloid, particularly isoquinoline alkaloids from plants such as Mahonia aquifolium.

U.S. Published patent application Ser. No. 20040131706 to Rittinghausen et al. describes the use of a pharmaceutical preparation for treating a variety of skin disorders comprising natural and/or synthetic active ingredient(s) extracted from plants such as Centella asiatica, Mahonia aquifolium, and Viola tricolor.

U.S. Published patent application Ser. No. 20020164386 by Meisner describes formulations for the treatment of psoriasis and related skin ailments comprising glucosamine in an emollient base such as a moisturizing cream. The formulations can further comprise keratolytic substances such as coal tar extract, salicylic acid, or antioxidant anti-inflammatory herbal extracts such as oleuropein and berberine.

Analogs of vitamin D3 metabolites have also been used to treat Psoriasis. The most well-known analogs are calcipotrol, which is sold under the brand name Dovonex®, and tacalcitol, which is sold under the brand name Curatoderm®. Vitamin D3 analogs are generally well tolerated with the most common side effect being irritation of the skin at the site of application (the vitamin D3 analogues are topical drugs, available as ointments, creams or a scalp solution in the case of Dovonex®). Studies have indicated that up to 20% of patients experience this side effect. The risk and severity of the side effects is increased dramatically when the drug is occluded, or covered, and thus drugs such as Dovonex® and Curatoderm® are not recommended for “skin fold” areas, where skin can occlude other skin. These drugs are also not recommended for the fact, where the skin is particularly sensitive.

There exists a need for compositions for the treatment of skin disorders that exhibit minimal side effects.

It is therefore an object of the invention to provide pharmaceutical compositions, which exhibit minimal side effects, for the treatment of skin disorders such as psoriasis, acne, rosacea, eczema (atopic dermatitis) and other types of dermatitis (eg, contact dermatitis, dyshidrotic eczema, nummular dermatitis, seborrheic dermatitis), verruca vulgaris, tuberous sclerosis, pyogenic granulomas, recessive dystrophic epidermolysis bullosa, venous ulcers, molluscum contagious, seborrheic keratosis, and actinic keratosis.

### BRIEF SUMMARY OF THE INVENTION

Compositions for the treatment of skin disorders comprising psorberine, an alcohol-water extract isolated from the Mahonia aquifolium plant, and one or more additional active agents, such as vitamin D3 analogs, antimicrobial agents, antifungal agents, corticoid steroids, antiseptic agents, skin protecting agents, retinoids, and local anesthetics or antihistamines are described herein. In a preferred embodiment, a vitamin D3 analog, such as calcipotriol, is included in the formulation. The compositions may also contain excipients such as emollients, surfactants, emulsifiers and buffers. The compositions may be formulated into ointments, creams, gels, lotions, powders, sprays, foams, shampoos for topical administration to treat skin disorders including psoriasis, acne, rosacea, eczema (atopic dermatitis) and other types of dermatitis (e.g, contact dermatitis), verruca vulgaris, tuberous sclerosis, pyogenic granulomas, recessive dystrophic epidermolysis bullosa, venous ulcers, molluscum contagious, seborrheic keratosis, and actinic keratosis.

### DETAILED DESCRIPTION OF THE INVENTION

I. Compositions

a. Psorbrine

Psorberine is an alcohol-water extract from the Mahonia Aquifolium tree. The Mahonia aquifolium extract is obtained in a highly concentrated form from crude dried Mahonia aquifolum, which is obtained from dried bark and twigs of plants from the Mahonia aquifolium family.

Mahonia aquifolium (Barberry, Oregon hollygrape, Berberis) belongs to the Berberidaceae family and grows wild in Europe and North and South America. Mahonia aquifolium has been used as a medication for inflammatory skin diseases such as psoriasis (Weisenauer, M. Z Allg. Med. (16:23-31 (1992); Gieler et al. J. Dermatol. Treatment (United Kingdom 6(1): 31-34 (1995)). The root and bark of the Mahonia aquifolum plant are known to contain isoquinoline alkaloids that include berberine, palmatine, berbamine, oxyacanthine, jatrorrhizine, bervulcine, magnoflorine and columbamine. These alkaloids are thought to be the active constituents of the plants, as many of them have shown strong in vitro anti-microbial and anti-fungal activity.

Mahonia aquifolium may have several mechanisms of action in the treatment and management of psoriasis and other inflammatory conditions. Hyper proliferation of keratinocytes is a major symptom of psoriasis and so controlling this activity will assist in the treatment of psoriasis. Laboratory studies have shown that berberine, the primary alkaloid isolated from Mahonia aquifolium, inhibits keratinocyte growth in vitro (Muller et al. Planta Medica 61(1): 74-75 (1995). Schmeller et al. (1997) demonstrated that berberine inhibits DNA synthesis by intercalculating into DNA and blocking the action of reverse transcriptase (Schmeller et al. Phytochemistry 44(2): 257-266 (1997)). Further studies demonstrated that topical application of Mahonia aquifolium reduced the inflammatory and keratinocyte hyperproliferation markers typically seen in psoriasis (Augustin et al. Zeutschraft Phtotherapie 17:44-45 (1996)).

The anti-psoriatic effects of Mahonia aquiflolium have been attributed to the primary alkaloid extracted from this plant, berberine. The anti-inflammatory effects of berberine have been linked to the inhibition of lipoxygenase and lipid peroxidation (Muller, K and Ziereis, K. Plants Medica 60(5); 421-4241 (1994); Bezakova et al. Pharmazie 51(10): 758-761 (1996); Misik et al. Planta Medica 61: 372-373 (1995)), and the cyclooxygenase pathway through the reduction of prostaglandin E2 (Kuo, Cancer Lett. 203(2): 127-137 (2004)). More recent evidence indicates berberine may inhibit the ability of cytokines to promote the inflammatory response (Davidson, A and Diamond, B. N. Engl. J. Med. 345:340-350 (2001); Hajnicka et al. Planta Med 68:226-268 (2002)). In addition, Mahonia aquifolium is a moderate inhibitor of LTB-4 (a leukotriene which is believed to mediate inflammation) and 5-hydroxy-eicosatetraenoic acid (5-HETE). All of these pathways are believed to contribute to the inflammation associated with psoriasis.

Psorberine also appears to have antiangiogenic activity and inhibits Interleukin-8 (IL-8) secretion by THP-1 cells treated with lipopolysaccharide (LPS). Studies have suggested that IL-8 may be important in psoriasis, as it is expressed in the stratum granulosum, attracts plymorphonuclear cells, and stimulates angiogenesis and keratinocyte mitogenesis (Konstantinova et al. J Invest Dermatol, 107(4):615-21 (1996)). Another study demonstrated that media conditioned by keratinocytes from psoriatic patients, including both symptomless skin and psoriatic plaques, induced vigorous angiogenic responses in over 90% of corneas tested and potently stimulated directional migration of capillary endothelial cells in vitro. The keratinocytes from the psoriatic skin exhibited a 10- to 20-fold increase in interleukin-8 production (Nickoloff BJ et al. Am J Pathol. 144(4):820-8 (1994)). Therefore, psorberine may be able to inhibit psoriasis and other skin diseases associated with angiogenesis through the inhibition of IL-8.

b. Other Active Agents
I. Vitamin D3 analogs

Suitable vitamin D3 analogues are described in U.S. Pat. No. 4,866,048 to Calverly et al. and have the general formula shown below: 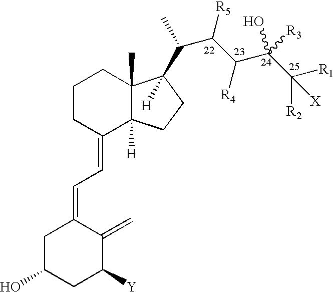
wherein X is a hydrogen, lower alkyl, halogen, or hydroxy; Y is a hydrogen or hydroxy; R₁ and R₂, which may be the same or different, are lower alkyl, optionally substituted with halogen or hydroxy with the proviso that R₁ and R₂ cannot both be methyl when X is other than lower aklyl; or, taken together with C25, R₁ and R₂ can form a saturated or unsaturated C₂-C₉ carbocyclic ring which may be optionally substituted at any possible position(s) with lower aklyl, halogen, or hydroxy; R₃ is a hydrogen or lower aklyl; R₄ and R₅ represent either a hydroge, or when taken together constitute a bond, with the result being that a double bond exists between C22 and C23. The expression “lower aklyl” indicates a straight or branched saturated or unsaturated carbon chain having from 1 to 8 carbons.

In a preferred embodiment, the vitamin D3 analog is calcipotriol, which has the structure shown below: 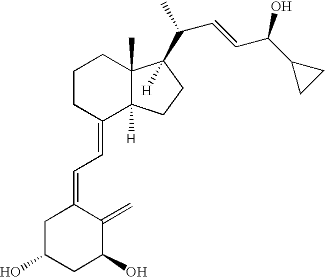
ii. Antimicrobial and Antifungal Agents

Antibacterial agents can be included in the compositions. A list of antibacterial agents is found in “Martindale—The Complete Drug Reference”, 32nd Ed., Kathleen Parfitt, (1999) on pages 112-270. Classes of useful antibacterials include, but are not limited to, aminoglycosides, antimycobacterials, cephalosporins and beta-lactams, chloramphenicols, glycopeptides, lincosamides, macrolides, penicillins, quinolones, sulphonamides and diaminopyridines, tetracyclines, clindamycin and other miscellaneous antimicrobial agents. In a preferred embodiment, triclosan is used in the topical formulations.

Alternatively or in addition to antimicrobial agents, antifungal agents can be included in the compositions. A list of anti-fungal agents can be found in “Martindale —The Complete Drug Reference”, 32nd Ed., Kathleen Parfitt, (1999) on pages 367-389. Suitable antifungals include, but are not limited to, amphotericin, amorolfine, bifonazole, bromochlorosalicyanilide, buclosamide, butenafine, butoconazole, candicidin, chlordantoin, chlormidazole, chlorphenesin, chlorxylenol, ciclopirox olamine, cilofungin, clotrimazole, croconazole, eberconazole, econazole, enilconazole, fenticlor, fenticonazole, fluconazole, flucytosine, griseofulvin, hachimycin, haloprogin, hydroxystilbamine, isethionate, iodochlorohydroxyquinone, isoconazole, itraconazole, ketoconazole, lanoconazole, luflucarban, mepartricin, miconazole, naftifine, natamycin, neticonazole, nifuroxime, nystatin, omoconazole, oxiconazole, pentamycin, propionic acid, protiofate, pyrrolnitrin, ravuconazole, saperconazole, selenium sulfide, sertaconazole, sulbenmtine, sulconazole, terbinafine, terconazole, tioconazole, tolciclate, tolnaftate, triacetin, timidazole, undeceonic acid, voriconazole and combinations thereof.

In a preferred embodiment, the anti-fungal agent(s) is an azole. Suitable imidazole and triazole antifungal agents include, but are not limited to, fluconazole, timidazole, secnidazole, miconazole nitrate, econazole, haloprogin, metronidazole, itraconazole, terconazole, posaconazole, ravuconazole, ketoconazole, clotimazole, sapirconazole and combinations thereof.
iii. Corticosteroids

Topical corticosteroids can also be included in the compositions. Examples of corticosteroids include, but are not limited to, betamethasone valerte or propionate, clobetasol propionate, desonide, dexamethasone sodium phosphate, fluocinolone acetonide, mometasone furoate, hydrocortisone, methylprednisolone acetate, mometasone furoate or triamcinolone acetonide.
iv. Antiseptic Agents

Antiseptic agents can be included in compositions formulated for topical administration. Suitable antiseptic agents include, but are not limited to, iodine, iodophores, chlorhexidine, gluconate, thimerosol, hydrogen peroxide, and abenzoyl peroxide.
v. Skin Protectants

Skin protectants can be included in compositions formulated for topical administration. Such agents not only soothe the skin but may also aide in maintaining the integrity of the skin to prevent additional damage. Suitable skin protectants include, but are not limited to, allantoin, cocoa butter, dimethicone, kaolin, shark liver oil, petrolatum, lanolin, vegetable oils, ethoxylated oils and lipids, polyalkylene oxides, polyvinylpyrrolidone, polyvinyl alcohol, polysaccharides, and zinc oxide.
vi. Local Anesthetics or Antihistamines

Local anesthetics or antihistamines may also be included in the topical formulation in order to lessen the pain and itching caused by the local infection. Suitable local anesthetics and antihistamines include, but are not limited to, benzocaine, lidocaine, dibucaine, etidocaine, benzyl alcohol, camphor, resorcinol, menthol, and diphenhdramine hydrochloride.
vii. Retinoids

Retinoids may also be included in the psorberine topical formulations. The retinoids are a family of compounds including vitamin A, retinoic acid (RA), related derivatives of these, and other compounds capable of binding to retinoic acid receptors (RAR). Many retinoids are known and have been described to date. Generally, retinoids can be identified by their ability to bind RARs, either as all the RARs or selectively to an individual RAR class. Examples of retinoids that may be used can be found in U.S. Pat. Nos. 6,048,902 to Lebwohl et al.; 4,476,056 to Pawson; 4,105,681 to Bollag et al.; 4,215,215 to Bollag et al.; 4,054,589 Bollag et al. and 3,882,244 to Lee. Preferred retinoids include tretinoin (marketed under the brand names Retin-A™, Retin-A Micro™, and Renova™ by OrthoNeutrogena), adapalene (marketed under the brand name Differin™ by Galderma Laboratories), and tazarotene (marketed under the brand name Tazorac™ by Allergan, Inc.).
viii. Azelaic Acid

Finacea™ (azelaic acid) Gel, 15%, contains azelaic acid, a naturally occurring saturated dicarboxylic acid (U.S. Pat. No. 4,713,394, Berlex Laboratories). Chemically, azelaic acid is 1,7-heptanedicarboxylic acid, with the molecular formula C₉H₁₆O₄ and a molecular weight of 188.22. The composition further contains an aqueous gel base containing benzoic acid (as a preservative), disodium-EDTA, lecithin, medium-chain triglycerides, polyacrylic acid, polysorbate 80, propylene glycol, purified water, and sodium hydroxide to adjust pH. Finacea™ Gel, 15%, is indicated for topical treatment of inflammatory papules and pustules of mild to moderate rosacea.
c. Excipients

Formulations may be prepared using pharmaceutically acceptable excipients composed of materials that are considered safe and effective and may be administered to an individual without causing undesirable biological side effects or unwanted interactions. The excipients are all components present in the pharmaceutical formulation other than the active ingredient or ingredients. As generally used herein “excipient” include, but is not limited to surfactants, emulsifiers, emulsion stabilizers, emollients, buffers, solvents and preservatives.
i. Emollients

Suitable emollients include those generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petolatum, petrolatum and lanolin alcohols, soybean oil,starach,stearyl alcohol, sunflower oil, xylitol and combinations thereof. In one embodiment, the emollients are ethylhexylstearate and ethylhexyl palmitate.
ii. Surfactants

Suitable non-ionic surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.
iii. Emulsifiers

Suitable emulsifier include, but are not limited, acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, snflower oil, tragacanth, triethanolamine, xanthan gum and combinations thereof. In one embodiment, the emulsifier is glycerol stearate.
iv. Buffers

Buffers preferably buffer the composition from a pH of about 4 to a pH of about 7.5, more preferably from a pH of about 4 to a pH of about 7, and most preferably from a pH of about 5 to a pH of about 7.

II. Method of Making

a. Psorberine

Psorberine is isolated from the Mahonia aquifolium plant using an alcohol and water extraction process. In a preferred embodiment, crude Mahonie aquifolium, water and alcohol are loaded into a stainless steel reactor vessel. The vessel is clamped shut. A pressure of 3 to 6 psi (volume dependent) is applied to the reactor vessel and the mixture is heated to a temperature not higher than 50° C., preferably about 40° C., while the contents are mixed with an internal counter-rotating agitating mixer. When the mixture reaches 40° C., an internal grinding mixer is engaged and the mixture is processed at a speed of about 3000 rpm in combination with the internal counter-rotating mixer for three hours. The mixture is kept at a pressure of 3 to 6 psi during mixing. After mixing for three hours, the mixture is allowed to cool for at least 24 hours at 3 to 6 psi. The cycle of mixing for 3 hours and cooling for 24 hours at 3 to 6 psi is repeated two more times.

After the third 24 hour cooling period, the pressure is released in the reactor and the reaction mixture is filtered through a coarse mesh filter and then through a 5 micron filter. The mixture is then placed under vacuum and heated to a temperature between 40° C. and 50° C., while mixing, to reduce and remove the solvents, until the mixture is approximately 6% of its original volume. The resultant product is re-filtered through a 1-micron filter.

This extraction process yields a finished Mahonic aquifolium extract with a concentration of approximately 1.5 mg/ml berberine alkaloid. A typical alcohol based extraction process yields a finished extract with a concentration of approximately 0.09 mg/ml berberine alkaloid.

III. Methods of Treatment

A. Disorders to be Treated

Skin diseases or conditions that may be treated include psoriasis, acne, rosacea, exzema (atopic dermatitis) and other types of dermatitis (eg, contact dermatitis, dyshidrotic eczema, nummular dermatitis, seborrheic dermatitis), verruca vulgaris, tuberous sclerosis, pyogenic granulomas, recessive dystrophic epidermolysis bullosa, venous ulcers, molluscum contagious, seborrheic keratosis, and actinic keratosis.

Psoriasis and a number of other skin diseases have been associated with the undesirable or pathological growth of new blood vessels, or angiogenesis. Under normal physiological conditions, humans or animals only undergo angiogenesis in very specific situations. For example, angiogenesis is normally observed in wound healing, fetal and embryonal development and formation of the corpus luteum, endometrium and placenta. The control of angiogenesis is a highly regulated system of angiogenic stimulators and inhibitors. The control of angiogenesis has been found to be altered in certain skin disease states, such as psoriasis, and, in many cases, the pathological damage associated with the disease is related to the uncontrolled angiogenesis. Therefore, therapies directed at the control or inhibition of angiogenesis could lead to the abrogatiaon or mitigation of these skin diseases.

B. Formulations

The formulations may be administered topically in dosage unit formulations containing conventional nontoxic pharmaceutically acceptable carriers, adjuvants, and vehicles as desired.

The compounds, or pharmaceutically acceptable salts thereof, can be formulated as pharmaceutical compositions, including their polymorphic variations. Such compositions can be administered topically in dosage unit formulations containing conventional nontoxic pharmaceutically acceptable carriers, adjuvants, and vehicles as desired. Topical administration may also involve the use of transdermal administration such as transdermal patches or iontophoresis devices.

Formulation of drugs is discussed in, for example, Hoover, John E., Remington's Pharmaceutical Sciences, Mack Publishing Co., Easton, Penna. (1975), and Liberman, H. A. and Lachman, L., Eds., Pharmaceutical Dosage Forms, Marcel Decker, New York, N.Y. (1980). The term “pharmaceutically acceptable salt” means those salts which retain the biological effectiveness and properties of the compounds used in the present invention, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary and tertiary amines, substituted amines including naturally-occurring substituted amines, and cyclic amines, including isopropylamine, trimethylamine, diethylamine, triethylamine, tripopylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that oather carboxylic acid derivatives, for example carboxylic acid amides, including carboxamides, lower alkyl carboxamides, di(lower alkyl) carboxamides, could be used.

The compounds (or pharmaceutically acceptable salts thereof) may be administered per se or in the form of a pharmaceutical composition wherein the active compound(s) is in admixture or mixture with one or more pharmaceutically acceptable carriers, excipients or dilutents. Pharmaceutical compositions may be formulated in a conventional manner using one or more physiologically acceptable carriers comprising excipients and auxiliaries which facilitate processing of the active compounds into preparations which can be used pharmaceutically.

The amount of active ingredient that can be combined with the carrier materials to produce a single dosage form will vary depending upon the patient and the particular mode of administration.

For topical application, the compound is combined with a carrier so that an effective dosage is delivered, based on the desired activity, at the site of application. The topical composition can be applied to the skin for treatment of diseases such as psoriasis. The carrier may be in the form of an ointment, cream, gel, shampoo, paste, foam, aerosol, suppository, pad or gelled stick. A topical composition for use of an ointment or gel consists of an effective amount of compound in an ophthalmically acceptable excipient such as buffered saline, mineral oil, vegetable oils such as corn or arachis oils, petroleum jelly, Miglyol 182 alcohol solutions, or liposomes or liposome-like products.

The formulation may be in the form of a modified, delayed, extended or pulsatile release dosage form. A modified release dosage form is one for which the drug release characteristics of time course and/or location are chosen to accomplish therapeutic or convenience objectives not offered by conventional dosage forms such as solutions, conventional ointments, or promptly dissolving dosage forms. Delayed release, extended release, and pulsatile release dosage forms and their combinations are types of modified release dosage forms. A delayed release dosage form is one that releases a drug (or drugs) at a time other than promptly after administration. An extended release dosage form is one that allows at least a twofold reduction in dosing frequency as compared to that drug presented as a conventional dosage form.

C. Dosages

The psorberine formulation is administered as required to alleviate the symptoms of the disorder. Assays can be performed to determine an effective amount of the agent, either in vitro or in vivo. Representative assays are described in the examples provided below. Other methods are known to those skilled in the art, and can be used to determine an effective dose of these and other agents for the treatment and prevention of diseases or other disorders as described herein.

For the treatment of skin disorders, the compounds are administered topically or regionally. In a preferred embodiment, the compounds are administered in an ointment, salve or other pharmaceutically acceptable carrier. The preferred means of administration is to apply the formulation topically, such as an ointment, lotion, gel, spray, powder, shampoo, or solution, in an amount effective to alleviate the symptoms, for example, between 0.01-10%, preferably 0.01-5%, and most preferably 0.01% to 2.5% of the compounds, administered 1-3 times daily, for a period of time effective to alleviate the symptoms of the disorder, preferably 0-6 months, or until clinical improvement of the disorder is noted. This can be measured in decreased redness, decreased thickness of the plaques, decreased scaling, decreased area of involvement, and/or clearing of plaques. In addition, in a skin disorder such as psoriasis, the compounds may be delivered for a period of time to improve the severity PASI (psoriasis area and severity index) score.

The present invention will be further understood by reference to the following non-limiting examples.

### EXAMPLE 1

### Minimum Inhibitory Concentration (MIC) Determination of Psorberine on Propionibacterium acnes

Serial dilutions of Psorberine representing concentrations of 50, 25, 12.5, 6.25, 3.13, 1.56, 0.78, 0.39 and 0.19%, were incubated with 5×10⁶ Propionibacterium acnes (ATT #6919) cells/ml. The minimum concentration at which 24 h growth was inhibited (MIC) was assessed by measuring the turbidity of the bacterial cultures.

The minimum inhibitory concentration (MIC) of psorberine on P. acnes was determined to be 1:3.13.

### EXAMPLE 2

### Effects of Psorberine on PMN H₂O₂ Generation

A polymorphonuclear (“PMN”) cell population was isolated via density gradient centrifugation on Ficoll-Hypaque from heparinized whole blood. The cells were washed with RPMI cell medium and then resuspended in KPRG (145 mM NaCl, 5.7 mM sodium phosphate, 4.86 mM KCl, 0.54 mM CaCl², 1.2 mM MgSO4, 5.5 mM glucose, pH 7.3) at a concentration of 1.5×10⁶ cells/ml. Cells (30,000 in 200 μl) were aliquoted to the wells of a 96 well culture plate and 25 μl of serially diluted Psorberine added. Twenty five μl of 1 μg/ml of lipopolysaccharide (“LPS”) in the solvent KPRG was then added and the cells were cultured for 1 h at 37° C. 50 μl of each culture supernatant was transferred to a new plate and the peroxide present quantitated with the Amplex Red Hydrogen peroxide/Peroxidase Kit from Molecular Probes.

Treatment of PMN with Psorberine did not significantly alter the level of peroxide released form LPS-stimulated PMNs (Table 1). Inclusion of 1:10-40 dilutions of Psorberine with non-stimulated PMN appeared to potentiate the generation of superoxide from non-stimulated cells. However, results obtained with 1:10, 1:20, and, to much lower extent, 1:40 dilutions, may not be reliable (see below).

TABLE 1
Effect of Psorberine on Peroxide Release from PMNs

| | | | | μM | |
|---|---|---|---|---|---|
| | OD | OD | Mean | (H2O2) | Std Dev. |
| W/out | | | | | |
| LPS | | | | | |
| No Compd | 0.038 | 0.034 | 0.036 | 0.17561 | 0.013797 |
| dil 1/10 | 0.067 | 0.083 | 0.075 | 0.365854 | 0.055189 |
| dil 1/20 | 0.075 | 0.074 | 0.0745 | 0.363415 | 0.003449 |
| dil 1/40 | 0.06 | 0.068 | 0.064 | 0.312195 | 0.027594 |
| dil 1/80 | 0.051 | 0.051 | 0.051 | 0.24878 | 0 |
| dil 1/160 | 0.038 | 0.034 | 0.036 | 0.17561 | 0.013797 |
| W/LPS | | | | | |
| No Cmpd | 0.066 | 0.063 | 0.0645 | 0.314634 | 0.010348 |
| dil 1/10 | 0.068 | 0.067 | 0.0675 | 0.329268 | 0.003449 |
| dil 1/20 | 0.057 | 0.06 | 0.0585 | 0.285366 | 0.010348 |
| dil 1/40 | 0.054 | 0.053 | 0.0535 | 0.260976 | 0.003449 |
| dil 1/80 | 0.051 | 0.066 | 0.0585 | 0.285366 | 0.05174 |
| Dil 1/160 | 0.063 | 0.06 | 0.0615 | 0.3 | 0.0103418 |

### EXAMPLE 3

### MTT Analysis/Cytokine Generation

THP-1 cells were sub-cultured in 96 well culture plates at a density of 20,000 cells/well. LPS (100 ng/ml) and titrating amounts of Psorberine and/or Calcipotriol were then added to triplicate wells. The cells were cultured for 5 days, at which point the culture plates were centrifuged, the culture media removed and saved, and fresh media containing 0.863 mg/ml MTT added. After culturing for an additional 4 h, the plates were centrifuged again, the media removed from the formazan crystals and the well contents solubilized in DMSO. The absorbance of each well at 560 nm was measured. The data were normalized to the averaged results of the control wells receiving water. The culture supernatants were tested in ELISA (Bio-Source, International) for IL-8, TNF-α and IL-1β content.

THP-1 cells were cultured in the presence of 100 ng/ml LPS and serial dilutions of Psorberine for 2 days and the effects on IL-8 release and proliferation were measured. IL-8 production was induced; nominal levels of IL-8 were detected in the culture supernatants of the non-stimulated cells (˜2-3 pg/ml), whereas 2.667+161 pg/ml was present after stimulation. The addition of Psorberine at all concentrations tested inhibited the induction of IL-8. It should be noted that 1:10 and 1:20 dilutions are cytolytic for THP-1 cells, and this effect may overshadow the inhibitory effects of the compound. However, higher dilutions (lower concentrations, i.e. 1:80, 1:160, 1:320, or higher) do not affect the viability of THP-1 cells (Table 2). Hence, the results obtained with these latter dilutions are more realistic. While LPS stimulation did not affect THP-1 proliferation, Psorberine had even less of an effect on THP-1 cell proliferation. Significant inhibition of signal in the MTT assay was only observed at the highest concentrations tested, i.e. dilutions of 1:10 and 1:20, which was most probably due to the non-specific killing of the cells, and the interference in the colorimetric assay.

TABLE 2
Viability of THP-1 Cells After Incubation with Psorberine

| | # Viable Cells | # Dead Cells | % Viability |
|---|---|---|---|
| No treatment | 38 | 8 | 83 |
| 1:10 Psorberine | 0 | 10 | 0 |
| 1:20 Psorberine | 2 | 15 | 12 |
| 1:40 Psorberine | 7 | 5 | 60 |
| 1:80 Psorberine | 8 | 4 | 66 |
| 1:160 Psorberine | 14 | 7 | 66 |
| 1:320 Psorberine | 16 | 4 | 80 |

Psorberine Effects on the Secretion of IL-8, TNF-α or IL-1β from THP-1 after 2 and 5 days of Incubation.

THP-1 cells were cultured for 2 and 5 days in the presence or absence of 100 ng/ml LPS and serial dilutions of Psorberine and the effects on IL-8 (Table 3), TNF-α (Table 4), and IL-1β secretion were measured (Table 5). Psorberine completely inhibited the generation of IL-8 when THP-1 cells were cultured in its presence for 2 days at approximately 1:1000 dilution for non-stimulated cells, at approximately 1:320 for LPS-stimulated cells. As indicated above, the inhibitory effect of 1:10, 1:20, and to some extent 1:40, is most plausibly due to the cytolysis of the cells. However, significant inhibition of IL-8 secretion was also observed with 1:80, 1:160, 1:320 and 1:640 dilutions that do not affect the viability of THP-1 cells. In addition, Psorberine inhibited TNF-α secretion from LPS-stimulated cells at both time points. Maximal inhibition was ˜60% exhibited at dilutions up to 1:640.

TABLE 3
Effect of Psorberine on IL-8 Release by THP-1 Cells

| | OD (mean) | Std. Dev. | pg/ml | Std Dev. |
|---|---|---|---|---|
| W/out LPS | | | | |
| IL-8 (2 days) | | | | |
| No Cmpd | 0.262 | 0.006 | 152.2 | 3.485496 |
| dil 1/10 | 0.019 | 0.003 | 4.239 | 0.669316 |
| dil 1/20 | 0.02 | 0.001 | 4.6 | 0.23 |
| dil 1/40 | 0.021 | 0.001 | 4.828 | 0.229905 |
| dil 1/80 | 0.109 | 0.003 | 45.76 | 1.25945 |
| dil 1/160 | 0.139 | 0.002 | 64.15 | 0.923022 |
| dil 1/320 | 0.135 | 0.006 | 61.107 | 2.715867 |
| dil 1/640 | 0.131 | 0.003 | 58.9 | 1.348855 |
| dil 1/1280 | 0.205 | 0.032 | 108.9 | 16.99902 |
| W/LPS | | | | |
| IL-8 (2 days) | | | | |
| No Cmpd | 0.262 | 0.006 | 152.2 | 3.485496 |
| dil 1/10 | 0.019 | 0.003 | 4.239 | 0.669316 |
| dil 1/20 | 0.02 | 0.001 | 4.6 | 0.23 |
| dil 1/40 | 0.021 | 0.001 | 4.828 | 0.229905 |
| dil 1/80 | 0.109 | 0.003 | 45.76 | 1.25945 |
| dil 1/160 | 0.139 | 0.002 | 64.15 | 0.923022 |
| dil 1/320 | 0.135 | 0.006 | 61.107 | 2.715867 |
| dil 1/640 | 0.131 | 0.003 | 58.9 | 1.348855 |
| dil 1/1280 | 0.205 | 0.032 | 108.9 | 16.99902 |
| W/out LPS | | | | |
| IL-8 (5 days) | | | | |
| No | 0.749 | 0.016 | 640 | 13.67156 |
| compound | | | | |
| dil 1/10 | 0.017 | 0.002 | 3.498 | 0.411529 |
| dil 1/20 | 0.019 | 0.002 | 4.204 | 0.442526 |
| dil 1/40 | 0.023 | 0.002 | 5.388 | 0.468522 |
| dil 1/80 | 0.129 | 0.01 | 57.99 | 4.495349 |
| dil 1/160 | 0.118 | 0.021 | 51.3 | 9.129661 |
| dil 1/320 | 0.173 | 0.05 | 86.1 | 24.88439 |
| dil 1/640 | 1.25 | 0.069 | 1289 | 71.1528 |
| dil 1/1280 | 1.249 | 0.089 | 1289 | 91.85028 |
| W/LPS | | | | |
| IL-8 (5 days) | | | | |
| No | 0.885 | 0.176 | 804 | 159.8915 |
| compound | | | | |
| dil 1/10 | 0.035 | 0.007 | 9.016 | 1.8032 |
| dil 1/20 | 0.037 | 0.002 | 9.436 | 0.510054 |
| dil 1/40 | 0.047 | 0.012 | 13.1 | 3.344681 |
| dil 1/80 | 0.192 | 0.03 | 86.128 | 13.4575 |
| dil 1/160 | 0.23 | 0.066 | 109.6 | 31.45043 |
| dil 1/320 | 0.596 | 0.271 | 391.144 | 177.8524 |
| dil 1/640 | 1.692 | 0.142 | 1574 | 132.0969 |
| dil 1/1280 | 1.711 | 0.371 | 1599 | 346.7148 |

TABLE 4
Effects of Psorberine on TNF-α Secretion from THP-1 Cells

| | OD (mean) | Std. Dev. | pg/ml | Std Dev. |
|---|---|---|---|---|
| W/out LPS | | | | |
| TNF-α (2 | | | | |
| days) | | | | |
| No Cmpd | 0.109 | 0.017 | 33.3 | 5.193578 |
| dil 1/10 | 0.103 | 0.023 | 29.78 | 6.649903 |
| dil 1/20 | 0.122 | 0.062 | 42.8 | 21.75082 |
| dil 1/40 | 0.148 | 0.029 | 65.1 | 12.75608 |
| dil 1/80 | 0.128 | 0.012 | 47.75 | 4.476563 |
| dil 1/160 | 0.134 | 0.025 | 52.45 | 9.785448 |
| dil 1/320 | 0.136 | 0.007 | 53.94 | 2.776324 |
| dil 1/640 | 0.119 | 0.015 | 40.27 | 5.07605 |
| dil 1/1280 | 0.146 | 0.007 | 62.64 | 3.003288 |
| W/LPS | | | | |
| TNF-α (2 | | | | |
| days) | | | | |
| No Cmpd | 0.157 | 0.016 | 73.96 | 7.537325 |
| dil 1/10 | 0.106 | 0.001 | 31.289 | 0.295179 |
| dil 1/20 | 0.112 | 0.006 | 35.84 | 1.92 |
| dil 1/40 | 0.101 | 0.005 | 28.5 | 1.410891 |
| dil 1/80 | 0.1 | 0.001 | 28 | 0.28 |
| dil 1/160 | 0.12 | 0.0281 | 40.96 | 9.591467 |
| dil 1/320 | 0.112 | 0.011 | 35.6 | 3.496429 |
| dil 1/640 | 0.112 | 0.014 | 35.37 | 4.42125 |
| dil 1/1280 | 0.134 | 0.044 | 52.3 | 17.17313 |
| W/out LPS | | | | |
| TNF-α (5 | | | | |
| days) | | | | |
| No | 0.099 | 0.012 | 27.4 | 3.321212 |
| compound | | | | |
| dil 1/10 | 0.111 | 0.041 | 21.5 | 7.941441 |
| dil 1/20 | 0.097 | 0.024 | 16.39 | 4.055258 |
| dil 1/40 | 0.109 | 0.027 | 20.71 | 5.13 |
| dil 1/80 | 0.117 | 0.039 | 23.9 | 7.966667 |
| dil 1/160 | 0.105 | 0.015 | 19.16 | 2.737143 |
| dil 1/320 | 0.119 | 0.02 | 24.6 | 4.134454 |
| dil 1/640 | 0.168 | 0.034 | 49.2 | 9.957143 |
| dil 1/1280 | 0.147 | 0.007 | 37.6 | 1.790476 |
| W/LPS | | | | |
| TNF-α (5 | | | | |
| days) | | | | |
| No | 0.147 | 0.016 | 37.6 | 4.092517 |
| compound | | | | |
| dil 1/10 | 0.112 | 0.008 | 21.9 | 1.564286 |
| dil 1/20 | 0.095 | 0.014 | 15.8 | 2.328421 |
| dil 1/40 | 0.106 | 0.005 | 19.6 | 0.924528 |
| dil 1/80 | 0.101 | 0.002 | 17.8 | 0.352475 |
| dil 1/160 | 0.098 | 0.012 | 16.82 | 2.059592 |
| dil 1/320 | 0.105 | 0.01 | 19.138 | 1.822667 |
| dil 1/640 | 0.13 | 0.003 | 29.318 | 0.676569 |
| dil 1/1280 | 0.123 | 0.003 | 26.07 | 0.635854 |

Detectable levels of IL-1β were demonstrated in the supernatants of the 5 day culture supernatants of the THP-1 cells (Table 5). Psorberine inhibited IL-1β secretion from non-stimulated as well as LPS-stimulated cells at dilutions that do not affect the viability of these cells (1:80, 1:160 and 1:320). A modest decrease (approximately 40%) was observed for the LPS-stimulated population at dilutions up to 1:640.

TABLE 5
Effects of Psorberine on IL-1β Generation from THP-1 Cells

| | OD (mean) | Std. Dev. | pg/ml | Std Dev. |
|---|---|---|---|---|
| W/out LPS | | | | |
| IL-1β (5 | | | | |
| days) | | | | |
| No Cmpd | 0.045 | 0.019 | 32.461 | 13.70576 |
| dil 1/10 | 0.025 | 0.001 | 14.168 | 0.56672 |
| dil 1/20 | 0.032 | 0.011 | 20.8 | 7.15 |
| dil 1/40 | 0.032 | 0.01 | 20.4 | 6.375 |
| dil 1/80 | 0.031 | 0.005 | 19.6 | 3.16129 |
| dil 1/160 | 0.03 | 0.006 | 18.7 | 3.74 |
| dii 1/320 | 0.031 | 0.002 | 19.08 | 1.230968 |
| dil 1/640 | 0.032 | 0.002 | 20 | 1.25 |
| dil 1/1280 | 0.146 | 0.007 | 62.64 | 3.003288 |
| W/LPS | | | | |
| IL-1β (5 | | | | |
| days) | | | | |
| No Cmpd | 0.041 | 0.005 | 28.237 | 3.443537 |
| dil 1/10 | 0.024 | 0.001 | 13.84 | 0.576667 |
| dil 1/20 | 0.023 | 0.003 | 12.96 | 1.690435 |
| dil 1/40 | 0.029 | 0.001 | 17.69 | 0.61 |
| dil 1/80 | 0.029 | 0.001 | 17.69 | 0.61 |
| dil 1/160 | 0.026 | 0.004 | 15.3 | 2.353846 |
| dil 1/320 | 0.024 | 0.001 | 13.8 | 0.575 |
| dil 1/640 | 0.028 | 0.002 | 17.11 | 1.222143 |
| dil 1/1280 | 0.034 | 0.008 | 22.2 | 5.223529 |

### EXAMPLE 4

### Effects of Psorberine on THP-1 Cell Proliferation

Isolation of T Cells

Twenty ml aliquots of heparinized whole blood were subjected to density gradient centrifugation on Ficoll Hypaque. The buffy coat layers representing peripheral blood mononuclear cells (PBMCs) containing lymphocytes and monocytes were washed once, resuspended in RPMI 1640 containing 10% fetal calf sera and then depleted of B cells and monocytes with a T cell enrichment column (R&D Systems). The T cell preparations were centrifuged, resuspended in 5 ml of incomplete RPMI and counted using a hemocytometer.

T Cell Proliferation

Aliquots of each cell preparation (40,000 cells in 200 μl of incomplete RPMI) were placed in the wells of 96 well culture plates and cultured overnight at 37° C. under 5% CO₂. Twenty five μl of RPMI containing 1 μg/ml PMA and 8 μg/ml ionomycin. After culturing for 24 h, the plates were centrifuged at 1200 rpm for 10 min and the culture supernatants removed and saved. Two hundred μl of complete RPMI containing 0.86 mg/ml MTT was added to each well and the plates cultured for an additional 4 h. The plates were centrifuged again, the media removed and the well contents solubilized in 100% DMSO. The optical density at 560 nm of each well was determined and the averaged mean of the duplicate well calculated. The background OD560 value, determined from control wells receiving media containing no MTT, was subtracted from these values and the data normalized to that of the control wells (0 nM compound). IL-2 secretion by activated T cells was determined in the culture supernatants of these cells. The supernatants were assessed using the R&D Biosystems ELISA kit for human IL-2.

The effect of Psorberine on THP-1 cell proliferation was examined. Treatment of LPS-stimulated T cell populations with Psorberine did not significantly alter the proliferation of these cells 2 or 5 days post incubation (Table 6). The increase in the percentage of control seen in the MTT assay at the high concentrations of Psorberine (1:10 and 1:20 dilutions), is most probably due to the strong coloring of the compound (dark green). There is a very high OD recovered from MTT assay upon incubating HT-29 cells with these dilutions of Psorberine. These data suggest that results obtained with dilutions below 1:40 may not be reliable for two reasons: 1. the toxicity of the compound; and 2. the false-positive results obtained in the MTT assay due to the strong coloring. The effect of titrating levels of Calcipotriol on THP-1 cell proliferation was multi-phasic, with stimulation seen above concentrations of 1 nM.

TABLE 6
MTT of THP-1 Cells Treated with Psorberine

| | | | | | Bckgd | % of |
|---|---|---|---|---|---|---|
| | OD | OD | OD | Mean | Sub | Control |
| W/out LPS | | | | | | |
| Background | 0.046 | 0.046 | | | | |
| No Cmpd | 0.071 | 0.071 | 0.072 | 0.071333 | 0.025333 | 100.0013 |
| dil 1/10 | 0.089 | 0.087 | | 0.088 | 0.042 | 165.7917 |
| dil 1/20 | 0.082 | 0.083 | 0.077 | 0.080667 | 0.034667 | 136.8439 |
| dil 1/40 | 0.079 | 0.075 | 0.078 | 0.077333 | 0.031333 | 123.6858 |
| dil 1/80 | 0.077 | 0.076 | 0.071 | 0.074667 | 0.028667 | 113.1594 |
| dil 1/160 | 0.077 | 0.075 | 0.077 | 0.076333 | 0.030333 | 119.7384 |
| dil 1/320 | 0.074 | 0.072 | 0.075 | 0.073667 | 0.027667 | 109.212 |
| dil 1/640 | 0.07 | 0.081 | 0.08 | 0.077 | 0.031 | 122.37 |
| dil 1/280 | 0.071 | 0.071 | 0.072 | 0.071333 | 0.025333 | 100.0013 |
| W/LPS | | | | | | |
| No Cmpd | 0.081 | 0.076 | 0.075 | 0.077333 | 0.031333 | 100.0011 |
| dil 1/10 | 0.092 | 0.09 | 0.086 | 0.089333 | 0.043333 | 138.2993 |
| dil 1/20 | 0.093 | 0.091 | 0.081 | 0.088333 | 0.042333 | 135.1078 |
| dil 1/40 | 0.091 | 0.085 | 0.092 | 0.089333 | 0.043333 | 138.2993 |
| dil 1/80 | 0.073 | 0.079 | 0.081 | 0.077667 | 0.031667 | 101.0649 |
| dil 1/160 | 0.077 | 0.076 | 0.078 | 0.077 | 0.031 | 98.93722 |
| dil 1/320 | 0.078 | 0.078 | 0.082 | 0.079333 | 0.033333 | 106.3841 |
| dil 1/640 | 0.078 | 0.075 | 0.085 | 0.079333 | 0.033333 | 106.3841 |
| dil 1/1280 | 0.081 | 0.076 | 0.075 | 0.077333 | 0.031333 | 100.0011 |

TABLE 7
Effects of Psorberine on the Proliferation of T Cells as
Measured by the MTT Assay

| | | | | OD | | | |
|---|---|---|---|---|---|---|---|
| | | | OD | (Std. | Bckgd | % of | |
| | OD | OD | (mean) | (Dev.) | Sub | Control | |
| T Cells | | | | | | | |
| No cmpd − PMA/Ion | 0.100 | 0.121 | 0.111 | 0.015 | 0.065 | | |
| No cmpd + PMA/Ion | 0.103 | 0.110 | 0.107 | 0.005 | 0.061 | 99.18033 | 4.609555 |
| 1/10 | 0.240 | 0.276 | 0.258 | 0.025 | 0.212 | 347.541 | 34.2905 |
| 1/20 | 0.152 | 0.142 | 0.147 | 0.007 | 0.101 | 165.5738 | 7.964513 |
| 1/40 | 0.098 | 0.102 | 0.100 | 0.003 | 0.054 | 88.52459 | 2.503854 |
| 1/80 | 0.085 | 0.087 | 0.086 | 0.001 | 0.040 | 65.57377 | 1.078318 |
| 1/160 | 0.081 | 0.080 | 0.081 | 0.001 | 0.035 | 56.55738 | 0.496796 |
| 1/320 | 0.090 | 0.083 | 0.087 | 0.005 | 0.041 | 66.39344 | 3.7992 |
| 1/640 | 0.082 | 0.100 | 0.091 | 0.013 | 0.045 | 73.77049 | 10.31808 |
| 1/1280 | 0.096 | 0.096 | 0.096 | 0.000 | 0.050 | 81.96721 | 0 |
| W/LPS | | | | | | | |
| No cmpd − PMA/Ion | 0.100 | 0.120 | 0.110 | 0.014 | 0.064 | 104.918 | 13.48877 |
| No cmpd + PMA/Ion | 0.110 | 0.121 | 0.116 | 0.008 | 0.070 | 113.9344 | 7.672743 |
| 100.000 | 0.091 | 0.103 | 0.097 | 0.008 | 0.051 | 83.60656 | 7.313661 |
| 50.000 | 0.107 | 0.123 | 0.115 | 0.011 | 0.069 | 113.1148 | 11.12824 |
| 10.000 | 0.117 | 0.117 | 0.117 | 0.000 | 0.071 | 116.3934 | 0 |
| 5.000 | 0.101 | 0.087 | 0.094 | 0.010 | 0.048 | 78.68852 | 8.286986 |
| 1.000 | 0.096 | 0.117 | 0.107 | 0.015 | 0.061 | 99.18033 | 13.82866 |
| 0.500 | 0.106 | 0.117 | 0.112 | 0.008 | 0.066 | 107.377 | 7.49056 |
| 0.100 | 0.098 | 0.102 | 0.100 | 0.003 | 0.054 | 88.52459 | 2.503854 |
| 0.050 | 0.106 | 0.119 | 0.113 | 0.009 | 0.067 | 109.0164 | 8.907742 |

Psorberine effect on Peripheral blood T cell proliferation was also investigated. A difference in T cell proliferation between cells incubated with or without PMA plus ionomycin after 24 h incubation was not observed. Addition of high concentrations of Psorberine (1:10 dilution, and to a lesser extent 1:20 dilution), to T cells even in the absence of PMA plus ionomycin resulted in high ODs, and in a seemingly false-positive effects on T cell proliferation (Table 7). However, at 1:280 dilution, Psorberine enhanced T cell proliferation in the absence of any stimulus. Significant differences in T cell proliferation were not observed after addition of Calcipotriol to T cells in the absence or presence of PMS plus ionomycin (Table 7).

IL-2 release was not significantly induced by treatment of THP-1 with LPS. Further treatment with Psorberine did not significantly alter the IL-2 expressed in these cells. A slight increase in IL-2 release was observed in the titration curve centered at ˜5 nM Calcipotriol. Adding Psorberine to final concentrations of 1:80 and 1:1,280 inhibited this small enhancement induced by Calcipotriol. Similar to the above conclusions, the results obtained with 1:10 dilution are not reliable due the toxicity of the compound at this concentration. However, results obtained utilizing 1:80 and 1:280 dilutions represent the actual effect of the compound.

The release of IL-2 was also determined 24 h after stimulating peripheral blood T cells with PMA plus ionomycin, in the presence or absence of Psorberine and/or Calcipotriol. Results demonstrate that T cells activated with PMA plus ionomycin secrete more than 4 ng/ml of IL-2. Calcipotriol (between 0.01-10 nM) reduced this concentration by about 40-50%. On the other hand, Psorberine at 1:80 dilution completely abrogated the secretion of IL-2 by activated T cells. The results obtained with 1:10 dilution are not reliable, due to toxicity. At 1:640 dilution Psorberine significantly inhibited IL-2 secretion by T cells and it enhanced the results with Calcipotriol for inhibiting IL-2 secretion.

### EXAMPLE 5

### Chemotaxis Assay of Jurakt Cells

Jurakt cells ere grown in cultures in the presence of complete RPMI media. These cells were split one day prior to the assay. More than 99% viable cells were used in the chemotaxis assay. SDF-1α was purchased from Peprotech, Inc.

96 well plate chemotaxis chambers with a pore size of 8 μM (ChemoTx, Neuro Probe, Inc.) were used in this assay. In the lower wells 30 μl of either RPMI plus 0.1% BSA (control), or 30 μl containing 1 μg/ml SDF-1α (positive chemotaxis) were placed. In other wells, 24 μl containing 1 μg/ml SDF-1α was added to 6 μl of media containing the appropriate concentrations of Psorberine, Calcipotriol or their combination. In the upper wells, 20 μl of media containing 20,000 cells (1×106/ml) either alone or in combinations with the compounds were placed. After 2 h, the upper part of the filters was washed with coton swaps to remove the non-migrating cells, and the filters were fixed with absolute methanol for 3 minutes. After this, the filters were stained with 15% Giemsa stain for 7.5 min. The filters were washed three times with distilled water, and the cells migrating to the lower wells were counted under light micriscope. For control (cells migrating in the absence of the chemoattractant), 7 filters were used, whereas 4 filters were used in the experimental conditions. Migration Index (MI) was calculated by dividing the number of cells migrating in the experimental filters by the average number of cells migrating in the control filters.

The chemotaxis assay was corroborated by determining the viability of Jurkat cells. Viability of these cells either incubated in culture medium alone or with Psorberine or Calcipotriol under the same conditions as in the chemotaxis assay, was done by distinguishing dead from viable cells in the Trypan blue exclusion test. Viability of THP-1 cells was determined after incubating these cells with various concentrations of Psorberine for 4 h.

Results indicate that SDF-1α when used at 1 μg/ml is a robust in vitro chemotactic factor for Jurkat cells. Psorberine at 1:10 or 1:40 dilutions completely abrogated SDF-1α-induced Jurkat cell chemotaxis. At 1:80 dilution, Psorberine inhibited about 50% of SDF-1α effect. In contrast, different concentrations (between 20-160 nM) of Calcipotriol did not affect SDF-1α-induced Jurkat cell chemotaxis (Table 8). Combining Psorberine with Calcipotriol did not significantly affect the inhibitory effect of Psorberine. In fact, there is somewhat a higher migratory effect when 20 nM of Calcipotriol was added to 1:80 dilution of Psorberne, as compared to the effect of 1:80 Psorberine without the addition of Calcipotriol (Table 8).

TABLE 8
Effects of Psorberine and/or Calcipotriol on T-Cell Proliferation

| | | | OD | OD | Bckgd | % of | |
|---|---|---|---|---|---|---|---|
| | OD | OD | (mean) | (Std. Dev.) | Sub | Control | |
| 0.000 | 0.110 | 0.121 | 0.116 | 0.008 | 0.070 | 113.9344 | 7.672743 |
| No cmpd − PMA/ | 0.100 | 0.120 | 0.110 | 0.014 | 0.064 | 104.918 | 13.48877 |
| Ion | | | | | | | |
| No cmpd + PMA/ | 0.103 | 0.110 | 0.107 | 0.005 | 0.061 | 99.18033 | 4.609555 |
| Ion | | | | | | | |
| 1/10 Ps | 0.240 | 0.276 | 0.258 | 0.025 | 0.212 | 347.541 | 34.2905 |
| 1/10 Ps + 0.1 nM | 0.259 | 0.252 | 0.256 | 0.005 | 0.210 | 343.4426 | 6.653441 |
| Cal | | | | | | | |
| 1/10 Ps + 1.0 nM | 0.249 | 0.255 | 0.252 | 0.004 | 0.206 | 337.7049 | 5.685558 |
| Cal | | | | | | | |
| 1/10 Ps + 10 nM | 0.266 | 0.247 | 0.257 | 0.013 | 0.211 | 345.082 | 18.0748 |
| Cal | | | | | | | |
| 1/80 Ps | 0.085 | 0.087 | 0.086 | 0.001 | 0.040 | 65.57377 | 1.078318 |
| 1/80 Ps + 0.1 nM | 0.089 | 0.081 | 0.085 | 0.006 | 0.039 | 63.93443 | 4.254914 |
| Cal | | | | | | | |
| 1/80 Ps + 1.0 nM | 0.086 | 0.085 | 0.086 | 0.001 | 0.040 | 64.7541 | 0.535533 |
| Cal | | | | | | | |
| 1/80 Ps + 10 nM | 0.084 | 0.081 | 0.083 | 0.002 | 0.037 | 59.83607 | 1.538563 |
| Cal | | | | | | | |
| 1/640 Ps | 0.082 | 0.100 | 0.091 | 0.013 | 0.045 | 73.77049 | 10.31808 |
| 1/640 Ps + 1.0 nM | 0.069 | 0.081 | 0.075 | 0.008 | 0.029 | 47.54098 | 5.378648 |
| Cal | | | | | | | |
| 1/640 Ps + 10 nM | 0.090 | 0.089 | 0.090 | 0.001 | 0.044 | 71.31148 | 0.563406 |
| Cal | | | | | | | |
| T Cells | | | | | | | |
| No cmpd − PMA/ | 0.108 | 0.093 | 0.101 | 0.011 | 0.055 | 99.09091 | 10.45789 |
| Ion | | | | | | | |
| No cmpd + PMA/ | 0.126 | 0.122 | 0.124 | 0.003 | 0.078 | | 0 |
| Ion | | | | | | | |
| 1/10 | 0.287 | 0.300 | 0.294 | 0.009 | 0.248 | 450 | 14.09395 |
| 1/20 | 0.141 | 0.168 | 0.155 | 0.019 | 0.109 | 197.2727 | 24.3774 |
| 1/40 | 0.101 | 0.121 | 0.111 | 0.014 | 0.065 | 118.1818 | 15.05715 |
| 1/80 | 0.091 | 0.084 | 0.088 | 0.005 | 0.042 | 75.45455 | 4.268354 |
| 1/160 | 0.095 | 0.088 | 0.092 | 0.005 | 0.046 | 82.72727 | 4.475182 |
| 1/320 | 0.086 | 0.087 | 0.087 | 0.001 | 0.041 | 73.63636 | 0.601951 |
| 1/640 | 0.101 | 0.109 | 0.105 | 0.006 | 0.059 | 107.2727 | 5.779297 |
| 1/1280 | 0.150 | 0.146 | 0.148 | 0.003 | 0.102 | 185.4545 | 3.544221 |

The viability of Jurkat cells incubated with Psorberine or Calcipotriol was examined under the same conditions as in the chemotaxis assay. Results in Table 9 demonstrate that Psorberine at 1:10 or 1:40 dilutions affected. Jurkat cell viability, which may contribute to the chemotaxis effect. However, this may not be the only factor contributing to the inhibitory activity of Psorberine, since 1:10 and 1:40 dilutions completely abrogated SDF-1α-induced Jurkat cell chemotaxis, but they only reduced the viability of these cells by 30% and 50%, respectively (Table 9).

TABLE 9
Effect of Psoberine and Calcipotriol or Their Combination
on the In Vitro Chemotaxis of the JURKAT Cell Line

| | Cell Number (MI) | | | | | | |
|---|---|---|---|---|---|---|---|
| | 1 | 2 | 3 | 4 | 5 | 6 | 7 |
| Control | 30 | 50 | 45 | 20 | 16 | 50 | 80 |
| 1 μg/ml | 200(4.8) | 210(5.0) | 190(4.5) | 90(2.1) | | | |
| SDF- | | | | | | | |
| 1α(CXCL12) | | | | | | | |
| 1 μg/ml | 5(0.2) | 5(0.2) | 4(0.1) | 25(0.6) | | | |
| SDF- | | | | | | | |
| 1α(CXCL12) + | | | | | | | |
| 1:10 | | | | | | | |
| Psorberine | | | | | | | |
| 1 μg/ml | 30(0.7) | 14(0.3) | 10(0.2) | 25(0.6) | | | |
| SDF- | | | | | | | |
| 1α(CXCL12) + | | | | | | | |
| 1:40 | | | | | | | |
| Psorberine | | | | | | | |
| 1 μg/ml | 134(3.2) | 52(1.2) | 100(2.4) | 60(1.4) | | | |
| SDF- | | | | | | | |
| 1α(CXCL12) + | | | | | | | |
| 1:80 | | | | | | | |
| Psorberine | | | | | | | |
| 1 μg/ml | 240(5.7) | 200(4.8) | 155(3.7) | 170(4.1) | | | |
| SDF- | | | | | | | |
| 1α(CXCL12) + | | | | | | | |
| 20 nM | | | | | | | |
| Calcipotriol | | | | | | | |
| 1 μg/ml | 240(5.7) | 160(3.8) | 180(4.3) | 220(5.2) | | | |
| SDF- | | | | | | | |
| 1α(CXCL12) + | | | | | | | |
| 80 nM | | | | | | | |
| Calcipotriol | | | | | | | |
| 1 μg/ml | 200(4.8) | 198(4.7) | 150(3.6) | 205(4.9) | | | |
| SDF- | | | | | | | |
| 1α(CXCL12) + | | | | | | | |
| 160 nM | | | | | | | |
| Calcipotriol | | | | | | | |
| 1 μg/ml | 2(0.1) | 4(0.1) | 4(0.1) | 10(0.2) | | | |
| SDF- | | | | | | | |
| 1α(CXCL12) + | | | | | | | |
| 1:20 | | | | | | | |
| Psorberine + 160 nm | | | | | | | |
| Calcipotriol | | | | | | | |
| 1 μg/ml | 4(0.1) | 4(0.1) | 4(0.1) | 30(0.7) | | | |
| SDF- | | | | | | | |
| 1α(CXCL12) + | | | | | | | |
| 1:40 | | | | | | | |
| Psorberine + 80 nm | | | | | | | |
| Calcipotriol | | | | | | | |
| 1 μg/ml | 148(3.5) | 120(5.9) | 130(3.1) | 155(3.7) | | | |
| SDF- | | | | | | | |
| 1α(CXCL12) + | | | | | | | |
| 1:80 | | | | | | | |
| Psorberine + 20 nm | | | | | | | |
| Calcipotriol | | | | | | | |

TABLE 10
Viability of JURKAT Cells Incubated with Psorberine or
Calcipotriol

| | # Viable Cells | # Dead Cells | % Viability |
|---|---|---|---|
| No treatment | 2 | 78 | 97.5 |
| 1:10 Psorberine | 99 | 51 | 34 |
| 1:40 Psorberine | 75 | 50 | 40 |
| 1:80 Psorberine | 57 | 67 | 54 |
| 20 nM | 18 | 178 | 91 |
| Calcipotriol | | | |
| 80 nM | 17 | 175 | 91 |
| Calcipotriol | | | |
| 160 nM | 11 | 134 | 92 |
| Calcipotriol | | | |

It is understood that the disclosed invention is not limited to the particular methodology, protocols, and reagents described above. Those skilled in the art will recognize, or be able to ascertain using no more than routine experimentation, equivalents to the specific embodiments of the invention described herein. Such equivalents are intended to be encompassed by the following claims. All references are specifically incorporated by reference herein.

1. A topical pharmaceutical composition comprising an effective amount of psorberine in combination with one or more additional pharmaceutically active agents for the treatment of a skin disorder or disease.

2. The composition of claim 1, wherein the combination is in an amount effective to treat a disease or disorder selected from the group consisting of psoriasis, acne, rosacea, eczema, contact dermatitis, dyshidrotic eczema, nummular dermatitis, seborrheic dermatitis, verruca vulgaris , tuberous sclerosis, pyogenic granulomas, recessive dystrophic epidermolysis bullosa, venous ulcers, molluscum contagious, seborrheic keratosis, and actinic keratosis.

3. The composition of claim 1, wherein the one or more active agents is selected from the group consisting of vitamin D3 analogs, antimicrobial agents, antifungal agents, corticoid steroids, antiseptic agents, skin protecting agents, retinoids, and triclosan.

4. The composition of claim 3, wherein the one or more active agents is a vitamin D3 analog.

5. The composition of claim 4, wherein the vitamin D3 analog has the general structure shown below: 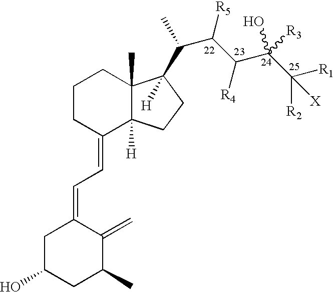
wherein X is a hydrogen, lower alkyl, halogen, or hydroxy; Y is a hydrogen or hydroxy; R₁ and R₂, which may be the same of different, are lower alkyl, optionally substituted with halogen or hydroxy with the proviso that R₁ and R₂ cannot both be methyl when X is other than lower alkyl; or, taken together with C25, R₁ and R₂ can form a saturated or unsaturated C₃-C₉ carbocyclic ring which may be optionally substituted at any possible position(s) with lower alkyl, halogen, or hydroxy; R₃ is a hydrogen or lower alkyl; R₄ and R₅ represent either a hydrogen, or when taken together constitute a bond, with the result being that a double bond exists between C22 and C23.

6. The composition of claim 5, wherein the vitamin D3 analog is calcipotriol.

7. The composition of claim 5, wherein the vitamin D3 analog is tacalcitol.

8. The composition of claim 3, wherein the one or more active agents is an antimicrobial agent.

9. The composition of claim 8, wherein the antimicrobial agent is clindamycin.

10. The composition of claim 3, wherein the one or more active agents is an antifungal agent.

11. The composition of claim 10, wherein the antifungal agent is metronidazole.

12. The composition of claim 1, further comprising an excipient.

13. The composition of claim 12, wherein the excipient is selected from the group consisting of emollients, surfactants, emulsifiers and buffers.

14. The composition of claim 1, wherein the composition is in a form selected from the group consisting of ointments, creams, gels, lotions, powders, sprays, foams, and shampoos.

15. The composition of claim 1, comprising 0.01 to 10% psorberine.

16. The composition of claim 15, comprising 0.01% to 2.5% psorberine.

17. A method of making a topical pharmaceutical composition comprising providing a composition comprising an effective amount of psorberine in combination with one or more additional pharmaceutically active agents for the treatment of a skin disorder or disease in a pharmaceutically acceptable carrier.

18. The method of claim 17, wherein the combination is in an amount effective to treat a disease or disorder s selected from the group consisting of psoriasis, acne, rosacea, eczema, contact dermatitis, dyshidrotic eczema, nummular dermatitis, seborrheic dermatitis, verruca vulgaris , tuberous sclerosis, pyogenic granulomas, recessive dystrophic epidermolysis bullosa, venous ulcers, molluscum contagious, seborrheic keratosis, and actinic keratosis.

19. The method of claim 17, wherein the one or more active agents is selected from the group consisting of vitamin D3 analogs, antimicrobial agents, antifungal agents, corticoid steroids, antiseptic agents, skin protecting agents, retinoids, and triclosan.

20. The method of claim 19, wherein the one or more active agents is a vitamin D3 analog.

21. The method of claim 20, wherein the vitamin D3 analog has the general structure shown below: 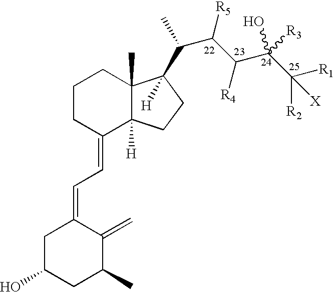
wherein X is a hydrogen, lower alkyl, halogen, or hydroxy; Y is a hydrogen or hydroxy; R₁ and R₂, which may be the same of different, are lower alkyl, optionally substituted with halogen or hydroxy with the proviso that R₁ and R₂ cannot both be methyl when X is other than lower alkyl; or, taken together with C25, R₁ and R₂ can form a saturated or unsaturated C₃-C₉ carbocyclic ring which may be optionally substituted at any possible position(s) with lower alkyl, halogen, or hydroxy; R₃ is a hydrogen or lower alkyl; R₄ and R₅ represent either a hydrogen, or when taken together constitute a bond, with the result being that a double bond exists between C22 and C23.

22. The method of claim 21, wherein the vitamin D3 analog is calcipotriol.

23. The method of claim 21, wherein the vitamin D3 analog is tacalcitol.

24. The method of claim 19, wherein the one or more active agents is an antimicrobial agent.

25. The method of claim 24, wherein the antimicrobial agent is clindamycin.

26. The method of claim 19, wherein the one or more active agents is an antifungal agent.

27. The method of claim 26, wherein the antifungal agent is metronidazole.

28. The method of claim 17, wherein the composition further comprises an excipient.

29. The method of claim 28, wherein the excipient is selected from the group consisting of emollients, surfactants, emulsifiers and buffers.

30. The method of claim 17, wherein the composition is in a form selected from the group consisting of ointments, creams, gels, lotions, powders, sprays, foams, and shampoos.

31. The method of claim 17, wherein the composition comprises 0.01 to 10% psorberine.

32. The method of claim 31, wherein the composition comprises 0.01% to 2.5% psorberine.

33. A method of treating a disease or disorder of the skin comprising applying to a site in need thereof a composition comprising an effective amount of psorberine in combination with one or more additional pharmaceutically active agents for the treatment of a skin disorder or disease.

34. The method of claim 33, wherein the combination is in an amount effective to treat a disease or disorder s selected from the group consisting of psoriasis, acne, rosacea, eczema, contact dermatitis, dyshidrotic eczema, nummular dermatitis, seborrheic dermatitis, verruca vulgaris , tuberous sclerosis, pyogenic granulomas, recessive dystrophic epidermolysis bullosa, venous ulcers, molluscum contagious, seborrheic keratosis, and actinic keratosis.

35. The method of claim 33, wherein the one or more active agents is selected from the group consisting of vitamin D3 analogs, antimicrobial agents, antifungal agents, corticoid steroids, antiseptic agents, skin protecting agents, retinoids, and triclosan.

36. The method of claim 35, wherein the one or more active agents is a vitamin D3 analog.

37. The method of claim 35, wherein the vitamin D3 analog has the general structure shown below: 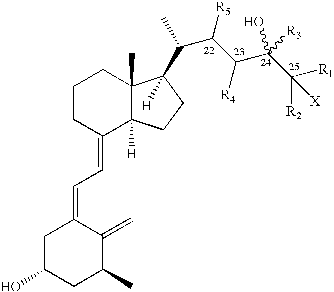
wherein X is a hydrogen, lower alkyl, halogen, or hydroxy; Y is a hydrogen or hydroxy; R₁ and R₂, which may be the same of different, are lower alkyl, optionally substituted with halogen or hydroxy with the proviso that R₁ and R₂ cannot both be methyl when X is other than lower alkyl; or, taken together with C25, R₁ and R₂ can form a saturated or unsaturated C₃-C₉ carbocyclic ring which may be optionally substituted at any possible position(s) with lower alkyl, halogen, or hydroxy; R₃ is a hydrogen or lower alkyl; R₄ and R₅ represent either a hydrogen, or when taken together constitute a bond, with the result being that a double bond exists between C22 and C23.

38. The method of claim 37, wherein the vitamin D3 analog is calcipotriol.

39. The composition of claim 37, wherein the vitamin D3 analog is tacalcitol.

40. The method of claim 35, wherein the one or more active agents is an antimicrobial agent.

41. The method of claim 40, wherein the antimicrobial agent is clindamycin.

42. The method of claim 35, wherein the one or more active agents is an antifungal agent.

43. The method of claim 42, wherein the antifungal agent is metronidazole.

44. The method of claim 33, wherein the composition further comprises an excipient.

45. The method of claim 44, wherein the excipient is selected from the group consisting of emollients, surfactants, emulsifiers and buffers.

46. The method of claim 33, wherein the composition is in a form selected from the group consisting of ointments, creams, gels, lotions, powders, sprays, foams, and shampoos.

47. The method of claim 33, wherein the composition comprises 0.01 to 10% psorberine.

48. The method of claim 47, wherein the composition comprises 0.01% to 2.5% psorberine.
